(19) United States
(12) Patent Application Publication
(10) Pub. No.: US 2011/0104100 A1
(43) Pub. Date: May 5, 2011

(54) COMPOSITIONS AND METHODS OF STEM CELL THERAPY FOR AUTISM

(75) Inventors: Neil H. Riordan, Tempe, AZ (US); Thomas E. Ichim, San Diego, CA (US)

(73) Assignee: MEDISTEM LABORATORIES, INC., San Diego, CA (US)

(21) Appl. No.: 12/681,600

(22) PCT Filed: Oct. 3, 2008
(86) PCT No.: PCT/US08/78852

§ 371 Date: Jan. 14, 2011

Related U.S. Application Data
(60) Provisional application No. 60/977,581, filed on Oct. 4, 2007.

(51) Int. Cl. A61K 38/19; A61K 35/12; A61K 35/14; A61K 35/34; A61K 35/50; A61K 35/32; A61K 38/18; A61P 25/00; A61P 43/00

(52) U.S. Cl. 424/85.1; 424/93.7; 514/7.7

(57) ABSTRACT

Disclosed are methods, compositions of matter, and cells, useful for the treatment of autism, social integrative disorders, and various cognitive abnormalities. The invention discloses, inter alia, means of substantially ameliorating or reversing the progression of autism through the administration of autologous and/or allogeneic stem cells, alone or in combination with mobilization agents. The use of stem cells and cells naturally possessing or endowed with angiogenic and anti-inflammatory activity are disclosed for autism either alone or in combination with various therapeutic interventions.

### RELATED APPLICATIONS

This application claims priority to U.S. Provisional Patent Application No: 60/977,581 filed on Oct. 4, 2007, the contents of which are hereby incorporated by reference in their entirety.

### BACKGROUND OF THE INVENTION

1. Field of the Invention

The invention pertains to the field of pervasive developmental disorders. More specifically, the invention pertains to the treatment of Autism, Rett's Disorder, Childhood Disintegrative Disorder, Asperger's Syndrome, and Pervasive Developmental Disorder Not Otherwise Specified (or PDDNOS) through administration of cellular therapies, as well as compounds capable of upregulating activities of said cellular therapies or mimicking activities of said cellular therapies.

2. Description of the Related Art

Autism is a highly prevalent disease believed to afflict approximately 1 in 155 children in the US. There is great need for therapies that are effective for the treatment of autism.

### SUMMARY OF THE INVENTION

Provided herein is the discovery centered around the concept that treatment of autism and autism spectrum disorders can be performed by ameliorating two main pathological features of this condition: hypoperfusion of specific areas of the brain, and inflammatory responses. Thus, as provided herein, by either sequentially, or concurrently inhibiting these processes it is feasible to induce reversal of the disease or ameliorate and minimize the disease.

Accordingly, provided herein is a method of treating a pervasive developmental disorder comprising: a) providing a cell with ability to inhibit host inflammatory reactions; b) providing an agent or therapy capable of mobilizing endogenous stem cells; and c) administering an effective amount of the cell and an effective amount of the agent or therapy; wherein the cell is administered prior to, subsequent to, or concurrent with the agent or therapy.

In certain embodiments, the pervasive developmental disorder can be for example: Autism, Rett's Disorder, Childhood Disintegrative Disorder, Asperger's Syndrome, and Pervasive Developmental Disorder Not Otherwise Specified (or PDDNOS). In selected embodiments, the disorder is autism. In certain embodiments, the cell with ability to inhibit host inflammatory reactions can be for example: a) a mesenchymal stem cell; b) an alternatively activated macrophage; c) a myeloid suppressor cell; and d) an immature dendritic cell. In certain embodiments, the cell with ability to inhibit host inflammatory reactions is autologous to the host. In certain embodiments, the cell with ability to inhibit host inflammatory reactions is allogeneic to the host.

In certain embodiments, the cells with ability to inhibit host inflammatory reactions are peripheral blood derived mesenchymal stem cells.

In certain embodiments, the agent capable of mobilizing endogenous stem cells can be for example: M-CSF, G-CSF, GM-CSF, an antagonist of CXCR-4, an antagonist of VLA-4, fucoidan, IVIG, parathyroid hormone, and cyclophosphamide.

In certain embodiments, the treatment capable of mobilizing endogenous stem cells can be selected from a group of treatments consisting of: hyperbaric oxygen, exercise, and autohemotherapy using extracorporeal ozonation.

Also provided herein is a method of treating a pervasive developmental disorder comprising: a) providing an agent with ability to inhibit host inflammatory reactions; b) providing an agent or therapy capable of mobilizing endogenous stem cells; and c) administering an effective amount of the agent with ability to inhibit host inflammatory reactions and an effective amount of the agent or therapy capable of mobilizing endogenous stem cells; wherein the agent with ability to inhibit host inflammatory reactions is administered prior to, subsequent to, or concurrent with the agent or therapy capable of mobilizing endogenous stem cells.

Also provided herein is a method of treating a pervasive developmental disorder comprising: a) providing a stem cell population; b) providing an agent or therapy capable of mobilizing endogenous stem cells; and c) administering an effective amount of the stem cell population and an effective amount of the agent or therapy capable of mobilizing endogenous stem cells; wherein the stem cell population is administered prior to, subsequent to, or concurrent with the agent or therapy.

Also provided herein is a method of treating a pervasive developmental disorder comprising: a) providing a stem cell population; b) providing an agent capable of stimulating proliferation of endogenous stem cells; and c) administering an effective amount of the stem cell population and an effective amount of the agent; wherein the stem cell population is administered prior to, subsequent to, or concurrent with the agent.

Also provided herein is a method of treating a pervasive developmental disorder comprising: a) providing an agent capable of stimulating proliferation of stem cells; b) providing an agent or therapy capable of mobilizing endogenous stem cells; and c) administering an effective amount of the agent capable of stimulating proliferation of stem cells and an effective amount of the agent or therapy capable of mobilizing the stem cells; wherein the agent capable of stimulating proliferation of stem cells is administered prior to, subsequent to, or concurrent with the agent or therapy capable of mobilizing the stem cells.

Also provided herein is a method of treating a pervasive developmental disorder comprising: providing an effective amount of an agent or plurality of agents capable of stimulating proliferation of stem cells administered at a concentration sufficient to ameliorate or reverse the pervasive developmental disorder.

Also provided herein is a method of treating a pervasive developmental disorder comprising: a) selecting a patient in need of treatment for a pervasive developmental disorder; and b) administering an effective amount of an agent or therapy capable of mobilizing stem cells at a concentration sufficient to ameliorate or reverse the pervasive developmental disorder.

Also provided herein is a method of treating a pervasive developmental disorder comprising: a) selecting a patient in need of treatment for a pervasive developmental disorder; and b) administering an effective amount of an agent or plurality of agents capable of stimulating proliferation of stem cells administered at a concentration sufficient to ameliorate or reverse the pervasive developmental disorder.

Also provided herein is a method of treating a pervasive developmental disorder comprising: a) selecting a patient in need of treatment for a pervasive developmental disorder; and b) administering an effective amount of stem cells to said patient, wherein said patient has undergone mobilization therapy.

In any of the above embodiments where an agent capable of mobilizing endogenous stem cells is administered, a range of effective concentrations can be used. For example, where G-CSF is administered, G-CSF can be administered at a concentration ranging from about 0.01, about 0.1, about 1 to about 5000, about 2000, about 1000, about 900, about 800, about 700, about 600, about 500, about 400, about 300, about 200, about 100, about 50, about 25, about 10, 9, 8, 7, 6, 5, 4, 3, or about 2 micrograms/kilogram of patient body weight per day for a period ranging from about 1 day to about 100 days. Other similar dose ranges are applicable for agent such as: M-CSF, G-CSF, GM-CSF, an antagonist of CXCR-4, an antagonist of VLA-4, fucoidan, IVIG, parathyroid hormone, and cyclophosphamide.

In any of the above embodiments, the amount of purified mononuclear cells administered can be at least 1, at least 10, at least 100, at least 1,000, at least 10,000, at least 100,000, at least 1 million, at least 10 million, or at least 100 million cells. The amount of cells administered can range from 1 cell to about 100 million cells, from about 100 cells to about 10 million cells, from about 1000 cells to about 10 million cells, from about 10,000 cells to about 10 million cells, from about 100,000 cells to about 10 million cells, from about 1 million cells to about 10 million cells, and from about 1 million cells to about 5 million cells.

### DETAILED DESCRIPTION OF THE PREFERRED EMBODIMENT

The invention provides means of treating autism through manipulation of the stem cell compartment and in some cases altering the chronic inflammatory mechanisms found in patients with autism.

Methods of addressing the issue of inflammation have included non-steroidal anti-inflammatory agents, corticosteroids, and PPAR modulating agents. Cellular therapies that possess anti-inflammatory properties include administration of various types of stem cells such as mesenchymal stem cells. Mesenchymal stem cells can suppress T-cell immunity.

Angiogenesis is known to involve circulating angioblast cells. One cellular population containing angioblast cells is the CD34 positive population. The inherent safety of non-related, unmatched CD34+ cells is known.

The processing of exogenous CD34 cells, as well as the various manipulation steps needed for ensuring high quality cells for infusion limit the ease of administering CD34 and mesenchymal stem cells for the treatment of autism. Several methods are disclosed for overcoming these drawbacks.

In accordance with the above, provided herein is the discovery centered around the concept that treatment of autism and autism spectrum disorders can be performed by ameliorating two main pathological features of this condition: hypoperfusion of specific areas of the brain, and inflammatory responses. Thus, as provided herein, by either sequentially, or concurrently inhibiting these processes it is feasible to induce reversal of the disease.

Accordingly, in one embodiment, cells with anti-inflammatory properties are administered to a patient in need thereof, said cells can be administered subsequently, concurrently, or prior to mobilization of autologous stem cells. Anti-inflammatory properties include ability of said cells to substantially downregulate production of mediators associated with a state of inflammation. Said mediators include cytokines, prostaglandins, leukotrienes, and various products of complement activation. Inflammation is also defined as a state of immune activation, associated with upregulation of the cytokine profile termed Th1 (Mosmann et al. Two types of murine helper T cell clone. I. Definition according to profiles of lymphokine activities and secreted proteins. J Immunol. 1986 Apr. 1; 136(7):2348-57). Cells with anti-inflammatory properties are well known in the art and could include immature dendritic cells (Mahnke et al. Tolerogenic dendritic cells and regulatory T cells: a two-way relationship. J Dermatol Sci. 2007 Jun; 46(3):159-67), monocytes that have been stressed ex vivo to endow properties of tolerogenic antigen presenting cells (Legitmo et al. In vitro treatment of monocytes with 8-methoxypsolaren and ultraviolet A light induces dendritic cells with a tolerogenic phenotype. Clin Exp Immunol. 2007 Jun; 148(3):564-72.), alternatively activated monocytes/macrophages (Gordon et al. Alternative activation of macrophages. Nat Rev Immunol. 2003 Jan. 3 (1):23-35), T regulatory cells (Bopp et al. Regulatory T cells—the renaissance of the suppressor T cells. Ann Med. 2007; 39(5):322-34), natural killer T cells (Nowak et al. Invariant NKT cells and tolerance. Int Rev Immunol. 2007 Jan-Apr; 26(1-2):95-119), Th2 cells, Th3 cells, and mesenchymal stem cells (Gotherstrom et al. Immunomodulation by multipotent mesenchymal stromal cells. Transplantation. 2007 Jul. 15; 84). Cells with anti-inflammatory activity may be autologous or allogeneic. Each of the references cited above is hereby incorporated by reference in its entirety.

In some embodiments of the invention, cells will be selected for anti-inflammatory activity. Assessment of the anti-inflammatory abilities of cells contemplated for use within the context of the current invention may be performed. Numerous methods are known in the art, for example they may include assessment of the putative anti-inflammatory cells to modulate immunological parameters in vitro. Putative anti-inflammatory cells may be co-cultured at various ratios with an immunological cell. Said immunological cell may be stimulated with an activatory stimulus. The ability of the putative anti-inflammatory cell to inhibit, in a dose-dependent manner, production of inflammatory cytokines or to augment production of anti-inflammatory cytokines, may be used as an output system of assessing anti-inflammatory activity. Additional output parameters may include: proliferation, cytotoxic activity, production of inflammatory mediators, or upregulation of surface markers associated with activation. Cytokines assessed may include: IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, TNF, IFN and RANKL.

In some embodiments said cells with anti-inflammatory activity require ex vivo expansion, and/or ex vivo treatment to endow properties associated with said anti-inflammatory state. For example, generation of anti-inflammatory mesenchymal stem cells useful for practice of the current invention may be performed through methods described for expansion and growth of said cells. U.S. Pat. No. 5,486,359 entitled “Human Mesenchymal Stem Cells” describes various methods of extracting, propagating, and identifying mesenchymal stem cells. U.S. Pat. No. 6,261,549 entitled “Human Mesenchymal Stem Cells from Peripheral Blood” teaches methods of extracting mesenchymal stem cells from circulating sources as opposed to bone marrow, which is usually associated with a certain degree of invasiveness. U.S. Pat. No. 6,368,636 entitled “Mesenchymal stem cells for prevention and treatment of immune responses in transplantation” teaches methods of harnessing anti-inflammatory activities of mesenchymal stem cell cells in the context of transplantation. These references serve as examples to one of skill in the art for the generation of mesenchymal stem cell populations useful within the context of the current invention. Each of the patents listed above is hereby incorporated by reference in its entirety.

In one particular embodiment mesenchymal stem cells are generated from the bone marrow of an autologous recipient. Bone marrow cells may be used in some embodiments that are allogeneic to the recipient. In other embodiments mesenchymal stem cells are generated from autologous peripheral blood sources. Methods of growing autologous mesenchymal stem cells are described in U.S. Pat. No. 6,261,549 entitled “Human Mesenchymal Stem Cells from Peripheral Blood”, which is incorporated herein by reference in its entirety.

Said cells inhibiting inflammation, or having potential to inhibit inflammation are co-administered, or administered prior to or subsequent to administration of stem cells. Said stem cells sources are numerous in the field and are listed below for exemplary purposes only. In one aspect of the invention said stem cells are selected from a group comprising of stem cells, committed progenitor cells, and differentiated cells. In a further aspect, said stem cells are selected from a group consisting of embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells. Selection of cells to be used in the practice of the invention is performed based on a number of relevant factors to the clinical utilization, including patient characteristics, availability of said cells, and need for immune suppression or other interventions when cells are administered.

In one particular embodiment the treatment is performed with the aim of mobilizing autologous CD34 cells so as to increase efficiency to home to the hypoperfused area of the brain and subsequently causing stimulation of angiogenesis and ultimately therefore decreasing hypoperfusion. The mesenchymal cells are incorporated into the treatment in order to inhibit the Th1 immune dysregulation systemically, and/or in some cases, specifically in the gut. The mobilization of CD34 cells is a procedure well known in the art. Specific descriptions of CD34 mobilization using G-CSF have been described (Heimfeld et al. Peripheral blood stem cell mobilization after stem cell factor or G-CSF treatment: rapid enrichment for stem and progenitor cells using the CEPRATE immunoaffinity separation system. Transplant Proc. 1992 Dec. 24 (6):2818; Freuhauf et al. Peripheral blood progenitor cell (PBPC) counts during steady-state hematopoiesis allow to estimate the yield of mobilized PBPC after filgrastim (R-metHuG-CSF)-supported cytotoxic chemotherapy. Blood. 1995 May 1, 85(9):2619-26, each of which is incorporated by reference in its entirety). Descriptions in the art exist of methods of mobilization tailored specifically for the pediatric population which may be useful in the practice of the current invention (see, for example, Kanold et al. CD34+ cell immunoselection from G-CSF-alone-primed peripheral blood in children with low body mass. Br J Haematol. 1995 Oct; 91(2):431-3). Methods of mobilizing stem cells without the use of G-CSF are widely known in the art. For example, mobilization can be achieved by the use of cyclophosphamide administration (Pierelli et al. Evaluation of a novel automated protocol for the collection of peripheral blood stem cells mobilized with chemotherapy or chemotherapy plus G-CSF using the Fresenius AS104 cell separator. J Hematother. 1993 Summer, 2(2):145-53), various chemotherapeutic agents (Brugger et al. Mobilization of peripheral blood progenitor cells by sequential administration of interleukin-3 and granulocyte-macrophage colony-stimulating factor following polychemotherapy with etoposide, ifosfamide, and cisplatin. Blood. 1992 Mar 1; 79(5):1193-200), CXCR4 antagonists (Hess et al. Human progenitor cells rapidly mobilized by AMD3100 repopulate NOD/SCID mice with increased frequency in comparison to cells from the same donor mobilized by granulocyte colony stimulating factor. Biol Blood Marrow Transplant. 2007 Apr; 13(4):398-411), seaweed extracts (Irhimeh et al. Fucoidan ingestion increases the expression of CXCR4 on human CD34+ cells. Exp Hematol. 2007 Jun; 35(6):989-94), exercise (Zaldivar et al. The effect of brief exercise on circulating CD34+ stem cells in early and late pubertal boys. Pediatr Res. 2007 Apr; 61(4):491-5), and hyperbaric oxygen (Thom et al. Stem cell mobilization by hyperbaric oxygen. Am J Physiol Heart Circ Physiol. 2006 Apr; 290(4):H1378-86). Each of the references in this paragraph is hereby incorporated by reference in its entirety. Mobilization of various types of stem cells using G-CSF is described in U.S. Pat. No. 7,220,407 which is incorporated herein by reference in its entirety.

Administration of immune suppressive cells, or cells with anti-inflammatory activity may be performed prior to, concurrently with, or subsequently after mobilization of endogenous stem cells. In some embodiments of the invention immunosuppressive cells are substituted for, or used together with agents known in the art to inhibit inflammation. Such agents include: Alclofenac; Alclometasone Dipropionate; Algestone Acetonide; Alpha Amylase; Alpha-lipoic acid; Alpha tocopherol; Amcinafal; Amcinafide; Amfenac Sodium; Amiprilose Hydrochloride; Anakinra; Anirolac; Anitrazafen; Apazone; Ascorbic Acid; Balsalazide Disodium; Bendazac; Benoxaprofen; Benzydamine Hydrochloride; Bromelains; Broperamole; Budesonide; Carprofen; Chlorogenic acid; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Ellagic acid; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Glutathione; Halcinonide; Halobetasol Propionate; Halopredone Acetate; Hesperedin; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Lycopene; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Oleuropein; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Pycnogenol; Polyphenols; Prednazate; Prifelone; Prodolic Acid; Proquazone; Proxazole; Proxazole Citrate; Quercetin; Reseveratrol; Rimexolone; Romazarit; Rosmarinic acid; Rutin; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrahydrocurcumin; Tetrydamine; Tiopinac; Tixocortol Pivalate; Tolmetin; Tolmetin Sodium; Triclonide; Triflumidate; Zidometacin; and Zomepirac Sodium.

In another embodiment, an exogenous angiogenic agent is administered systemically to alleviated the need for endogenous stem cell mobilization. Said agents stimulatory of angiogenesis may be administered together with mesenchymal stem cells. In this embodiment the mesenchymal stem cells inhibit inflammatory processes, whereas the exogenously administered angiogenic agent stimulates angiogenesis in order to increase perfusion. The use of exogenous angiogenic agents is preferably, but not exclusively, limited to agents that have specific activity on hypoxic tissue. In this manner angiogenesis will be limited to the area of hypoperfusion. Agents that selectively induce angiogenesis in areas of hypoperfusion include factors such as members of the FGF family whose receptors are upregulated in areas of tissue hypoxia.

In another embodiment angiogenesis stimulatory cells are provided together with an exogenous immune modulator. Such exogenous immune modulators may have anti-inflammatory activity such as IL-10, IL-4, or TGF family members. Other anti-inflammatory agents useful for the practice of this invention will be obvious to one of skill in the art. Examples of clinically used anti-inflammatory agents are known in the art and some have been provided previously.

As used herein, the term therapeutically effective amount refers to an amount or concentration which is effective in reducing, eliminating, treating, preventing or controlling the symptoms of a pervasive developmental disorder affecting a mammal. The term controlling is intended to refer to all processes wherein there may be a slowing, interrupting, arresting or stopping of the progression of the pervasive developmental disorder affecting the mammal. However, controlling does not necessarily indicate a total elimination of all disease and condition symptoms, and is intended to include prophylactic treatment.

In another aspect of the invention, patients with autism are treated with a therapeutically effective amount for treatment of a pervasive developmental disorder, of autologous cord blood stem cells together with said cell or agent possessing anti-inflammatory properties, said cord blood stem cells may be identified by expression of one or more markers selected from a group comprising: SSEA-3, SSEA-4, CD9, CD34, c-kit, OCT-4, Nanog, CD133 and CXCR-4, and lack of expression of markers selected from a group consisting of: CD3, CD45, and CD11b. In some aspects of the invention cord blood cells are used without purification by subset.

In another aspect of the invention, patients are treated with a therapeutically effective amount for treatment of a pervasive developmental disorder, of autologous placental stem cells, together with said cell or agent possessing anti-inflammatory activities said stem cells may be identified based on expression of one or more antigens selected from a group comprising: Oct-4, Rex-1, CD9, CD13, CD29, CD44, CD166, CD90, CD105, SH-3, SH-4, TRA-1-60, TRA-1-81, SSEA-4 and Sox-2. In some aspects of the invention placental stem cells are used without purification by subset.

In another aspect of the invention, patients are treated with a therapeutically effective amount for treatment of a pervasive developmental disorder, of autologous bone marrow stem cells together with cells or agents possessing anti-inflammatory properties; said bone marrow stem cells comprised of bone marrow derived mononuclear cells. Said bone marrow stem cells may also be selected based upon ability to differentiate into one or more of the following cell types: endothelial cells, muscle cells, and neuronal cells. Said bone marrow stem cells may also be selected based on expression of one or more of the following antigens: CD34, c-kit, flk-1, Stro-1, CD105, CD73, CD31, CD146, vascular endothelial-cadherin, CD133 and CXCR-4. In one particular aspect, said bone marrow stem cells are selectively enriched for mononuclear cells expressing the protein marker CD133.

In another aspect of the invention, patients are treated with a therapeutically effective amount for treatment of a pervasive developmental disorder, of autologous amniotic fluid stem cells along with said cell type or agent capable of inhibiting inflammation, wherein said amniotic fluid stem cells are isolated by introduction of a fluid extraction means into the amniotic cavity under ultrasound guidance. Said amniotic fluid stem cells may be selected based on expression of one or more of the following antigens: SSEA3, SSEA4, Tra-1-60, Tra-1-81, Tra-2-54, HLA class I, CD13, CD44, CD49b, CD105, Oct-4, Rex-1, DAZL and Runx-1 and lack of expression of one or more of the following antigens: CD34, CD45, and HLA Class II.

In another aspect of the invention, patients are treated with a therapeutically effective amount for treatment of a pervasive developmental disorder, of autologous neuronal stem cells together with said cell or agents possessing ability to inhibit inflammatory response, said neuronal stem cell are selected based on expression of one or more of the following antigens: RC-2, 3CB2, BLB, Sox-2hh, GLAST, Pax 6, nestin, Muashi-1, NCAM , A2B5 and prominin.

In another aspect of the invention, patients with are treated with a therapeutically effective amount for treatment of a pervasive developmental disorder, of autologous peripheral blood derived stem cells together with said cell or agent possessing anti-inflammatory activities. Said peripheral blood derived stem cells may be characterized by expression of one or more markers selected from a group comprising of CD34, CXCR4, CD117, CD113, and c-met, and in some cases by ability to proliferate in vitro for a period of over 3 months. In some situations peripheral blood stem cells are purified based on lack of expression of one or more differentiation associated markers, said one or more markers selected from a group comprising of CD2, CD3, CD4, CD11, CD11a, Mac-1, CD14, CD16, CD19, CD24, CD33, CD36, CD38, CD45, CD56, CD64, CD68, CD86, CD66b, and HLA-DR.

In another aspect of the invention, patients are treated with a therapeutically effective amount for treatment of a pervasive developmental disorder, of autologous mesenchymal stem cells together with said cell or agent possessing anti-inflammatory activity, said cells may be defined by expression of one or more of the following markers: STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, and in some situations lack of substantial levels of one or more of the following markers: HLA-DR, CD117, and CD45. In some aspects said mesenchymal stem cells are derived from a group selected of: bone marrow, adipose tissue, umbilical cord blood, placental tissue, peripheral blood mononuclear cells, differentiated embryonic stem cells, and differentiated progenitor cells.

In another aspect of the invention, patients are treated with a therapeutically effective amount for treatment of a pervasive developmental disorder, of autologous germinal stem cells together with said cell or agent capable of suppressing inflammatory responses, wherein said germinal stem cells may express one or more markers selected from a group consisting of: Oct4, Nanog, Dppa5 Rbm, cyclin A2, Tex18, Stra8, Daz1, beta1- and alpha6-integrins, Vasa, Fragilis, Nobox, c-Kit, Sca-1 and Rex1.

In another aspect of the invention, patients with are treated with a therapeutically effective amount for treatment of a pervasive developmental disorder, of autologous adipose tissue derived stem cells, together with said cell or agent possessing anti-inflammatory activity, wherein said adipose tissue derived stem cells may express one or more markers selected from a group consisting of: CD13, CD29, CD44, CD63, CD73, CD90, CD166, Aldehyde dehydrogenase (ALDH), and ABCG2. In an alternative aspect adipose tissue derived stem cells derived as mononuclear cells extracted from adipose tissue that are capable of proliferating in culture for more than 1 month. Cells capable of inhibiting inflammation are administered with said adipose derived stem cells.

In another aspect of the invention, patients are treated with a therapeutically effective amount for treatment of a pervasive developmental disorder, of autologous exfoliated teeth derived stem cells together with a cell or agent possessing anti-inflammatory activities, wherein said exfoliated teeth derived stem cells may express one or more markers selected from a group consisting of: STRO-1, CD146 (MUC18), alkaline phosphatase, MEPE, and bFGF.

In another aspect of the invention, patients are treated with a therapeutically effective amount for treatment of a pervasive developmental disorder, of autologous hair follicle stem cell together with a therapeutically sufficient concentration of cells or agents capable of activating antiinflammatory pathways, wherein said hair follicle stem cells may express one or more markers selected from a group consisting of: cytokeratin 15, Nanog, and Oct-4, in some aspects, said hair follicle stem cells are chosen based on capable of proliferating in culture for a period of at least one month. In other aspects, said hair follicle stem cell is selected based on ability to secrete one or more of the following proteins when grown in culture: basic fibroblast growth factor (bFGF), endothelin-1 (ET-1) and stem cell factor (SCF).

In another aspect of the invention, patients are treated with a therapeutically effective amount for treatment of a pervasive developmental disorder, of dermal stem cells and said cells or agents possessing anti-inflammatory properties, wherein said dermal stem cells express one or more markers selected from a group consisting of: CD44, CD13, CD29, CD90, and CD105. In some aspects, said dermal stem cells are chosen based on ability of proliferating in culture for a period of at least one month.

In another aspect of the invention, patients are treated with a therapeutically effective amount for treatment of a pervasive developmental disorder, of parthenogenically derived stem cells and said cell or agent possessing anti-inflammatory activities, wherein said parthenogenically derived stem cells are generated by addition of a calcium flux inducing agent to activate an oocyte followed by enrichment of cells expressing one or more markers selected from a group comprising of SSEA-4, TRA 1-60 and TRA 1-81.

In another aspect of the invention, patients are treated with a therapeutically effective amount for treatment of a pervasive developmental disorder, of stem cells generated by reprogramming and cells or agents possessing anti-inflammatory properties, said reprogramming being induced, for example, by nuclear transfer, cytoplasmic transfer, or cells treated with a DNA methyltransferase inhibitor, cells treated with a histone deacetylase inhibitor, cells treated with a GSK-3 inhibitor, cells induced to dedifferentiate by alteration of extracellular conditions, and cells treated with various combination of the mentioned treatment conditions.

In another aspect of the invention, patients are treated with a therapeutically effective amount for treatment of a pervasive developmental disorder, of autologous side population cells and cells or agents possessing anti-inflammatory properties, wherein said cells are identified based on expression multidrug resistance transport protein (ABCG2) or ability to efflux intracellular dyes such as rhodamine-123 and or Hoechst 33342. Said side population cells may be derived from tissues such as pancreatic tissue, liver tissue, muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue.

In another aspect of the invention, patients are treated with a therapeutically effective amount for treatment of a pervasive developmental disorder, of autologous committed progenitor cells and a cell type or agent capable of maintaining a state of suppressed inflammation, wherein said committed progenitor cells are selected from a group consisting of: endothelial progenitor cells, neuronal progenitor cells, and hematopoietic progenitor cells. In another aspect of the invention, committed progenitor cells are purified from peripheral blood of a patient whose committed endothelial progenitor cells are mobilized by administration of a mobilizing agent or therapy. Said mobilizing agent is selected from a group consisting of: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA) reductase inhibitors and small molecule antagonists of SDF-1. Said mobilization therapy is selected from a group consisting of: exercise, hyperbaric oxygen, autohemotherapy by ex vivo ozonation of peripheral blood, and induction of SDF-1 secretion in an anatomical area outside of the bone marrow.

In another aspect of the invention, patients are treated by enhancing the number of circulating stem cells in a patient in need thereof, said enhancement may be performed through administration of a mobilization agent, or mobilization therapy, said mobilizing agent may be selected from a group consisting of: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA) reductase inhibitors and small molecule antagonists of SDF-1. Said mobilization therapy may be selected from a group consisting of: exercise, hyperbaric oxygen, autohemotherapy by ex vivo ozonation of peripheral blood, and induction of SDF-1 secretion in an anatomical area outside of the bone marrow.

In another aspect of the invention, patients with autism in which resident stem cells have been mobilized by administration of a mobilizing agent are further treated with agent(s) capable of stimulating proliferation of endogenous stem cells. Such agents are known in the art and include prolactin; growth hormone, estrogen, ciliary neurotrophic factor (CNTF), pituitary adenylate cyclase activating polypeptide (PACAP), fibroblast growth factor (FGF), transforming growth factor alpha (TGF.alpha.), epidermal growth factor (EGF), erythropoietin, human chorionic gonadotrophin, cardiotrophin, IGF, thalidomide, valproic acid, G-CSF, trichostatin A, sodium phenylbutyrate, 5-azacytidine, and FSH. In one particular embodiment stem cell mobilization is achieved by administration of G-CSF, subsequent to which endogenous neurogenesis is stimulated by administration of prolactin in combination with EPO. Various methods of stimulating proliferation of endogenous stem cells are known in the art. Some are described in U.S. Pat. No. 7,048,934 entitled “Combined regulation of neural cell production”, and U.S. patent application No. 2003/0054998 entitled “Prolactin induced increase in neural stem cell numbers”, U.S. patent application No. 2003/0054551 entitled “Effect of growth hormone and IGF-1 on neural stem cells”, U.S. patent application No. 20070179092 entitled “Method of Enhancing Neural Stem Cell Proliferation, Differentiation, and Survival Using Pituitary Adenylate Cyclase Activating Polypeptide (PACAP)”, U.S. patent application No. 2007/0009491 entitled “Platelet-derived growth factor-responsive neural precursor cells and progeny thereof,” U.S. patent application No. 2005/0245436 entitled “Pheromones and the luteinizing hormone for inducing proliferation of neural stem cells and neurogenesis”, and U.S. patent application No. 2006/0089309 entitled “Stimulation of proliferation of pluripotential stem cells through administration of pregnancy associated compounds.” Each of the patents and patent applications listed above is hereby incorporated by reference in its entirety.

### Routes of Administration

Routes of administration of stem cells and agents capable of mobilizing endogenous stem cells are known in the art and may include parenteral, e.g., intravenous, intradermal, subcutaneous, oral (e.g., ingestion or inhalation), transdermal (topical), transmucosal, and rectal administration.

One simple method of administering stem cells is through the systemic route. Systemic administration of stem cells requires dilution of cells into appropriate solutions so that cells maintain viability. In one embodiment of the invention cells are administered in a solution of phosphate buffered saline, in another embodiment cells are dissolved in a solution of saline supplemented with autologous serum at a concentration ranging between 1-10%, preferably, between 2-7%, and even more preferably at a concentration of approximately 3%. It is known to one skilled in the art that various concentrations of albumin may also be added with the saline for injection of cells. Ideally pH of the injection solution should be from about 6.4 to about 8.3, optimally 7.4. Excipients may be used to bring the solution to isotonicity such as, 4.5% mannitol or 0.9% sodium chloride, pH buffers with art-known buffer solutions. Other pharmaceutically acceptable agents can also be used to bring the solution to isotonicity, including, but not limited to, dextrose, boric acid, sodium tartrate, propylene glycol, polyols (such as mannitol and sorbitol) or other inorganic or organic solutes.

Concentration and frequency of cellular administration is dependent on patient characteristics, as well as type of stem cells used. Numerous other factors may be used to guide the practitioner of the invention for adjusting the dose of stem cells administered. Said factors include the amount of endogenous stem cells circulating in the patient, the activity of stem cells in the patient (ie proliferative, colony formation, chemotactic mobility, etc), and the degree of the target indication that is observed in the patient.

### Injection Steps and Dose Ranges

In any of the above embodiments where an agent capable of mobilizing endogenous stem cells is administered, a range of effective concentrations can be used. For example, where G-CSF is administered, G-CSF can be administered at a concentration ranging from about 0.01, about 0.1, about 1 to about 5000, about 2000, about 1000, about 900, about 800, about 700, about 600, about 500, about 400, about 300, about 200, about 100, about 50, about 25, about 10, 9, 8, 7, 6, 5, 4, 3, or about 2 micrograms/kilogram of patient body weight per day for a period ranging from about 1 day to about 100 days. Other similar dose ranges are applicable for agent such as: M-CSF, G-CSF, GM-CSF, an antagonist of CXCR-4, an antagonist of VLA-4, fucoidan, IVIG, parathyroid hormone, and cyclophosphamide.

In any of the above embodiments, the amount of purified mononuclear cells administered can be at least 1, at least 10, at least 100, at least 1,000, at least 10,000, at least 100,000, at least 1 million, at least 10 million, or at least 100 million cells. The amount of cells administered can range from 1 cell to about 100 million cells, from about 100 cells to about 10 million cells, from about 1000 cells to about 10 million cells, from about 10,000 cells to about 10 million cells, from about 100,000 cells to about 10 million cells, from about 1 million cells to about 10 million cells, and from about 1 million cells to about 5 million cells.

In one embodiment of the invention, mononuclear cells are concentrated in an injection solution, which may be saline, mixtures of autologous plasma together with saline, or various concentrations of albumin with saline. Typically the pH of the injection solution is from about 6.4 to about 8.3, optimally 7.4. Excipients may be used to bring the solution to isotonicity such as, 4.5% mannitol or 0.9% sodium chloride, pH buffers with art-known buffer solutions, such as sodium phosphate. Other pharmaceutically acceptable agents can also be used to bring the solution to isotonicity, including, but not limited to, dextrose, boric acid, sodium tartrate, propylene glycol, polyols (such as mannitol and sorbitol) or other inorganic or organic solutes.

### EXAMPLES

### Example 1

### TREATMENT OF AUTISM USING AUTOLOGOUS MESENCHYMAL STEM CELLS AND MOBILIZATION.

Children with autism are recruited into an experimental study. About half of the patients serve as placebo controls whereas about half receive active treatment. The Aberrant Behavior Checklist (ABC) score and the Vineland Adaptive Behavior Scale are used in the selection of patients to enable the study to compare groups with similar characteristics. The treated group receives treatment comprising blood drawing, expansion of circulating mesenchymal stem cell progenitors and mobilization using G-CSF. The control groups have blood drawn but reinfusion is performed with saline. In the treated group mesenchymal cells are expanded and approximately 5 million cells are injected intravenously for a period of 10 days once every second day. Mobilization is performed by administration of G-CSF at a concentration of 9 micrograms per kilogram per day for a period of 3 days. Mobilization is initiated concurrently with the first infusion of autologous mesenchymal stem cells. One month after the last stem cell administration the ABC score and the Vineland Adaptive Behavior Scale are assessed. Significant improvements are seen in the treated patients but not controls.

### Example 2

### TREATMENT OF AUTISM BY STEM CELL MOBILIZATION AND INDUCTION OF NEUROGENESIS

Children with autism are recruited into an experimental study. About half of the patients serve as placebo controls whereas the other half of the patients receives active treatment. The Aberrant Behavior Checklist (ABC) score and the Vineland Adaptive Behavior Scale are used in the selection of patients to enable the study to compare groups with similar characteristics. Stem cell mobilization is induced by administration of G-CSF at a concentration of 9 micrograms per kilogram per day for a period of 3 days. The following day after the last injection of G-CSF, endogenous stem cells are stimulated to proliferate through systemic administration of hCG 3 times, once every second day at a dose at 10,000 RJ followed by administration of erythropoietin for 3 consecutive days at a concentration of 30,000 IU per dose. No significant adverse effects are noted during and subsequent to administration of stem cell mobilizer (G-CSF) and activators of endogenous stem cell proliferation (hCG and EPO). One month after the last stem cell administration the ABC score and the Vineland Adaptive Behavior Scale are assessed. Significant improvements are seen in the treated patients but not controls.

### Example 3

### TREATMENT OF AUTISM BY ALLOGENEIC CORD BLOOD MESENCHYMAL STEM CELLS AND MOBILIZATION OF ENDOGENOUS STEM CELLS

Children with autism are recruited into an experimental study. About half of the patients serve as placebo controls whereas half receive active treatment. The Aberrant Behavior Checklist (ABC) score and the Vineland Adaptive Behavior Scale are used in the selection of patients to enable the study to compare groups with similar characteristics.

Umbilical cord blood is purified according to routine methods (Rubinstein, et al. Processing and cryopreservation of placental/umbilical cord blood for unrelated bone marrow reconstitution. Proc Natl Acad Sci USA 92:10119-10122, incorporated herein by reference in its entirety). Briefly, a 16-gauge needle from a standard Baxter 450-ml blood donor set containing CPD A anticoagulant (citrate/phosphate/dextrose/adenine) (Baxter Health Care, Deerfield, Ill.) is inserted and used to puncture the umbilical vein of a placenta obtained from healthy delivery from a mother tested for viral and bacterial infections according to international donor standards. Cord blood was allowed to drain by gravity so as to drip into the blood bag. The placenta is placed in a plastic-lined, absorbent cotton pad suspended from a specially constructed support frame in order to allow collection and reduce the contamination with maternal blood and other secretions. The 63 ml of CPD A used in the standard blood transfusion bag, calculated for 450 ml of blood, is reduced to 23 ml by draining 40 ml into a graduated cylinder just prior to collection. An aliquot of the cord blood is removed for safety testing according to the standards of the National Marrow Donor Program (NMDP) guidelines. Safety testing includes routine laboratory detection of human immunodeficiency virus 1 and 2, human T-cell lymphotropic virus I and II, Hepatitis B virus, Hepatitis C virus, Cytomegalovirus and Syphilis. Subsequently, 6% (wt/vol) hydroxyethyl starch is added to the anticoagulated cord blood to a final concentration of 1.2%. The leukocyte-rich supernatant is then separated by centrifuging the cord blood hydroxyethyl starch mixture in the original collection blood bag (50×g for 5 min at 10° C.). The leukocyte-rich supernatant is expressed from the bag into a 150-ml Plasma Transfer bag (Baxter Health Care) and centrifuged (400×g for 10 min) to sediment the cells. Surplus supernatant plasma is transferred into a second plasma transfer bag without severing the connecting tube. Finally, the sedimented leukocytes are resuspended in supernatant plasma to a total volume of 20 ml. Approximately 5×108-7×109 nucleated cells are obtained per cord. Cord blood mononuclear cells are seeded at a density of 1×10⁶ cells/cm² into culture flasks in a Good Manufacturing Procedures-compliant sterile clean room. Cells are cultured in DMEM-LG media (Life Technologies), supplemented with 10% autologous serum. On day 4, nonadherent cells are discarded and fresh tissue culture medium is added. On day 7, cultures are tested for sterility, nonadherent cells are discarded by washing culture flasks with USP saline containing 10% autologous serum, and the remaining adherent cells are washed with Tyrode's Salt Solution (Sigma, St. Louis, Mo.) and incubated for 1 hr in M199 media (Life Technologies). Cells are detached with 0.05% trypsin-EDTA (Life Technologies), and are resuspended in MI99 supplemented with 10% of autologous serum. Cells are subcultured for 12 days with feeding of cultures performed every 3 days. The cells are subsequently harvested by trypsinization as described above, counted and an aliquot is taken for flow cytometric analyzes for the expression of mesenchymal stem cells markers and lack of expression of hematopoietic markers. Cell batches of >95% purity for CD73, and CD105, and less than 5% contamination of CD45 expressing cells are chosen for cell therapy.

Cord blood derived mesenchymal stem cells are adjusted to a concentration of 5×10⁷ cells in USP saline supplemented with 10% autologous serum and injected systemically in a volume of 50 ml in the period of 2 hours. Injection is performed once every two days for a total of 4 injections. Subsequent to the last injection patients are mobilized by administration of G-CSF at a concentration of 9 micrograms per kilogram per day for a period of 3 days.

One month after stem cell mobilization the ABC score and the Vineland Adaptive Behavior Scale are assessed. Significant improvements are seen in the treated patients but not controls.

One skilled in the art will appreciate that these methods, compositions, and cells are and may be adapted to carry out the objects and obtain the ends and advantages mentioned, as well as those inherent therein. The methods, procedures, and devices described herein are presently representative of preferred embodiments and are exemplary and are not intended as limitations on the scope of the invention. Changes therein and other uses will occur to those skilled in the art which are encompassed within the spirit of the invention and are defined by the scope of the disclosure. It will be apparent to one skilled in the art that varying substitutions and modifications may be made to the invention disclosed herein without departing from the scope and spirit of the invention. Those skilled in the art recognize that the aspects and embodiments of the invention set forth herein may be practiced separate from each other or in conjunction with each other. Therefore, combinations of separate embodiments are within the scope of the invention as disclosed herein. All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the invention disclosed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the disclosure.

### References.

1. World J Gastroenterol. 2006 Sep 21; 12(35):5606-10. Interleukin-12 and Th1 immune response in Crohn's disease: pathogenetic relevance and therapeutic implication. Peluso I, Pallone F, Monteleone G.

2 J Neuroimmunol. 2006 Apr; 173(1-2):126-34. Epub 2006 Feb 21. Immune activation of peripheral blood and mucosal CD3+ lymphocyte cytokine profiles in children with autism and gastrointestinal symptoms. Ashwood P, Wakefield A J.

3. J Neuroimmunol. 1996 May; 66(1-2):143-5 Plasma increase of interleukin-12 and interferon-gamma. Pathological significance in autism. Singh V K.

4. Ann Neurol. 2005 Sep; 58(3):466-9. Autism severity and temporal lobe functional abnormalities Gendry Meresse I,

5. http://www dot celltherapynews dot com/index.cfm?act=nl&do=newsletter&nl_ID=198&yr=2007&mnth=1

1. A method of treating a pervasive developmental disorder comprising:
a) selecting a patient in need of treatment for a pervasive developmental disorder;
b) administering an effective amount of an agent or therapy capable of mobilizing endogenous stem cells at a concentration sufficient to ameliorate or reverse said pervasive developmental disorder.

2. A method of treating a pervasive developmental disorder comprising:
a) selecting a patient in need of treatment for a pervasive developmental disorder;
b) administering an effective amount of an agent or plurality of agents capable of stimulating proliferation of stem cells administered at a concentration sufficient to ameliorate or reverse said pervasive developmental disorder.

3. A method of treating a pervasive developmental disorder comprising:
a) selecting a patient in need of treatment for a pervasive developmental disorder; and
b) administering an effective amount of a stem cell population to said patient, wherein said patient has undergone mobilization therapy.

4. A method of treating a pervasive developmental disorder comprising:
a) providing a cell with ability to inhibit host inflammatory reactions;
b) providing an agent or therapy capable of mobilizing endogenous stem cells; and
c) administering an effective amount of said cell and said agent or therapy;
wherein said cell is administered prior to, subsequent to, or concurrent with said agent or therapy.

5. The method of claim 1, wherein said pervasive developmental disorder is selected from a group consisting of: Autism, Rett's Disorder, Childhood Disintegrative Disorder, Asperger's Syndrome, and Pervasive Developmental Disorder Not Otherwise Specified (or PDDNOS).

6. The method of claim 5, wherein said disorder is autism.

7-10. (canceled)

11. The method of claim 1, wherein said agent capable of mobilizing endogenous stem cells is selected from a group consisting of: M-CSF, G-CSF, GM-CSF, an antagonist of CXCR-4, an antagonist of VLA-4, fucoidan, IVIG, parathyroid hormone, and cyclophosphamide.

12. The method of claim 1, wherein said treatment capable of mobilizing endogenous stem cells is selected from a group of treatments consisting of: hyperbaric oxygen, exercise, and autohemotherapy using extracorporeal ozonation.

13-15. (canceled)

16. The method of claim 4, wherein said agent with ability to inhibit host inflammatory reactions is selected from a group consisting of: a) a small molecule; b) a nucleic acid; c) a protein.

17-19. (canceled)

20. The method of claim 16, wherein said anti-inflammatory small molecule agent is selected from a group consisting of: pioglitazone, aspirin, ibuprofen, n-acetylcysteine, and resveratrol

21-25. (canceled)

26. The method of claim 3, wherein said stem cell population is autologous or allogeneic to said patient.

27. The method of claim 3, wherein said stem cell population is extracted from a group consisting of: a) peripheral blood; b) mobilized peripheral blood; c) adipose tissue; d) muscle tissue; e) bone marrow; f) placental tissue; g) cord blood; h) amniotic fluid; i) amnion; j) decidous teeth; and k) Warton's jelly.

28. The method of claim 27, wherein said stem cell population is a circulating mesenchymal stem cell.

29-36. (canceled)

37. The method of claim 2, wherein said agent capable of stimulating proliferation of stem cells is selected from one or more agents of a group consisting of: prolactin; growth hormone, estrogen, ciliary neurotrophic factor (CNTF), pituitary adenylate cyclase activating polypeptide (PACAP), fibroblast growth factor (FGF), transforming growth factor alpha (TGF.alpha.), epidermal growth factor (EGF), erythropoietin, human chorionic gonadotrophin, cardiotrophin, IGF, thalidomide, valproic acid, G-CSF, trichostatin A, sodium phenylbutyrate, 5-azacytidine, and FSH.

38-41. (canceled)

42. The method of claim 37, wherein G-CSF is administered at a concentration ranging from 1-200 micrograms/kilogram of patient body weight per day for a period ranging from 1 day to 100 days.

43. The method of claim 37, wherein erythropoietin is administered at a concentration ranging from 1000-100000 IU per day for a period ranging from 1 day to 100 days.

44. A method of treating a pervasive developmental disorder comprising:
a) providing an agent capable of stimulating proliferation of stem cells;
b) providing an agent or therapy capable of mobilizing endogenous stem cells; and
c) administering an effective amount of said agent capable of stimulating proliferation of stem cells and an effective amount of said agent or therapy capable of mobilizing said stem cells;
wherein said agent capable of stimulating proliferation of stem cells is administered prior to, subsequent to, or concurrent with said agent or therapy capable of mobilizing said stem cells.

45-65. (canceled)

66. The method of claim 3, wherein said stem cell population administered to said patient is in an amount ranging from about 10,000 cells to about 10 million cells.

67. The method of claim 3, wherein said stem cell population administered to said patient is in an amount ranging from about 100,000 cells to about 10 million cells.

68. The method of claim 3, wherein said stem cell population administered to said patient is in an amount ranging from about 1 million cells to about 10 million cells.
